(19) United States
(12) Patent Application Publication
(10) Pub. No.: US 2024/0180490 A1
(43) Pub. Date: Jun. 6, 2024

(54) HANDHELD LOCOMOTIVE STYLE AND PACE FEEDBACK SYSTEM

(71) Applicant: Christian SCHROEFL, Sydney (AU)

(72) Inventor: Christian SCHROEFL, Sydney (AU)

(21) Appl. No.: 18/551,820

(22) PCT Filed: Dec. 15, 2022
(86) PCT No.: PCT/AU2022/051515

§ 371 Date: Sep. 21, 2023

(30) Foreign Application Priority Data
Dec. 17, 2021 (AU) 2021904102

(51) Int. Cl. A61B 5/00

(52) U.S. Cl. CPC A61B 5/6826; A61B 5/7445; A61B 5/7455; A61B 5/7475

(57) ABSTRACT

A handheld locomotive style and pace feedback sys-tem comprises an electronic and a handheld device. The system is configured to operate in a configuration mode wherein the controllers of the electronic device control a user interface displayed by the digital display to set a locomotive style category and a pace setpoint and to transmit configuration settings to the handheld device accordingly. The system is further configured to operate in a feedback mode wherein the controllers of the handheld device measure accelerometer signals from the to determine a measured amplitude to compare the measured amplitude to an amplitude range of the locomotive style category to calculate a locomotive style comparison and to determine a measured period to compare the measured period to the pace setpoint to calculate a pace setpoint comparison. The handheld device controls the feedback device according to the locomotive style comparison and the pace setpoint comparison.

### FIELD OF THE INVENTION

This invention relates generally to a type of handheld exercise feedback system which is designed to provide locomotive style and pace feedback during walking or running.

### BACKGROUND OF THE INVENTION

Wearable technology includes activity trackers comprising sensors to measure exercise parameters, such as heart rate, motion, distance and the like to record exercise.

A common form of activity tracker is the smartwatch which is a wearable computer in the form of a watch.

US 2017/0361159 A1 (MALCOLM) 21 Dec. 2017 conversely discloses a modular activity tracker held in the hand.

Activity trackers typically comprise a digital display for a user interface which is used to configure the device and to display recorded parameters. Many activity trackers also comprise GPS receivers to measure distance.

The present invention seeks to provide a system which will overcome or substantially ameliorate at least some of the deficiencies of the prior art, or to at least provide an alternative.

It is to be understood that, if any prior art information is referred to herein, such reference does not constitute an admission that the information forms part of the common general knowledge in the art, in Australia or any other country.

### SUMMARY OF THE DISCLOSURE

There is provided herein a handheld locomotive style and pace feedback system which is designed to be ergonomic and reliable to provide feedback of a user's running or walking style and pace in a relatively distraction free manner.

A system comprises an electronic device comprising a processor, a digital display controlled by the processor, a data interface controlled by the processor and a memory device in operable communication with the processor, the memory device comprising computer program code instruction controllers executed by the processor.

The system further comprises a handheld device comprising a processor, a data interface controlled by the processor, an accelerometer in operable communication with the processor, a feedback device controlled by the processor, the processor executing computer program code instruction controllers

The system is configured to operate operates in a configuration mode wherein the controllers of the electronic device control a user interface displayed by the digital display to set a locomotive style category and pace setpoint.

The electronic device then transmits configuration settings according to the locomotive style category and the pace setpoint to the handheld device via the data interface of the handheld device.

The system is further configurable in a feedback mode wherein the controllers of the handheld device measure signals from the accelerometer to determine measured amplitude and measured period.

The controllers of the handheld device then compare the measured amplitude to an amplitude range accorded to of the locomotive style category to calculate a locomotive style comparison. The controllers of the handheld device further compare the measured period to the pace setpoint to calculate a pace setpoint comparison.

The controllers of the handheld device then control the feedback device according to the locomotive style comparison and the pace setpoint comparison.

The present system allows the convenient configuration of the handheld device using the electronic device, thereby give negating the need for complicated or distracting handheld device user interfaces. Once configured, the handheld device is able to reliably measure acceleration signals from the accelerometer to provide feedback relating to locomotive style and pace.

More specifically, the panel device may convert amplitude of accelerometer signals to in our range of motion and provide feedback as to whether the user is swinging the arms too much or too little with respect to the configured locomotive style. For example, arm motion range for a long-distance running locomotive style would be smaller than that of a sprinter locomotive style.

In embodiments, the system may be configured for measuring the distance between the pair of handheld devices to thereby provide further composition locomotive style feedback. For example, the arms for a sprinter locomotive style would be further apart either side of the torso as compared to that for a long-distance running locomotive style wherein the hands would generally be held close together in front of the chest. The system may measure distance between the handheld devices using a received signal strength indication or alternatively by analysing accelerometer signals, especially wherein the handheld devices comprise triaxial accelerometers and the phase of acceleration signals along the frontal axis is used to infer distance between the handheld devices.

The handle device may comprise a simple distraction free visual indicator comprising above range, in-range and below range LEDs for each of the locomotive style and pace feedback. The visual indicator may further comprise further LEDs to provide our position locomotive style feedback.

In embodiments, the handheld device may take the form of a body comprising a periphery of figure indentations which may be ergonomically held and which may provide proprioceptive input during running or walking.

The body may comprise a wide portion comprising three middle finger indentations and a narrow portion comprising opposing figure indentations for opposing grip of the outer fingers. The body may comprise an opening therethrough and may be flexible to be slightly squeezable.

The narrow portion may define a central portion between the outer fingers comprising a visual indicator.

Other aspects of the invention are also disclosed.

### BRIEF DESCRIPTION OF THE DRAWINGS

Notwithstanding any other forms which may fall within the scope of the present invention, preferred embodiments of the disclosure will now be described, by way of example only, with reference to the accompanying drawings in which:

FIG. 1 shows a functional schematic of feedback system in accordance with an embodiment;

FIG. 2 shows exemplary configuration mode processing by an electronic device of the system;

FIG. 3 shows exemplary feedback mode processing by an electronic device of the system;

FIG. 4 shows an exemplary handheld device;

FIG. 5 shows an exemplary configuration user interface;

FIG. 6 shows an exemplary visual indicator of the handheld device of the system in accordance with an embodiment;

FIG. 7 shows exemplary accelerometer signals measured by the handheld device; and

FIG. 8 illustrates holding of the handheld device.

### DESCRIPTION OF EMBODIMENTS

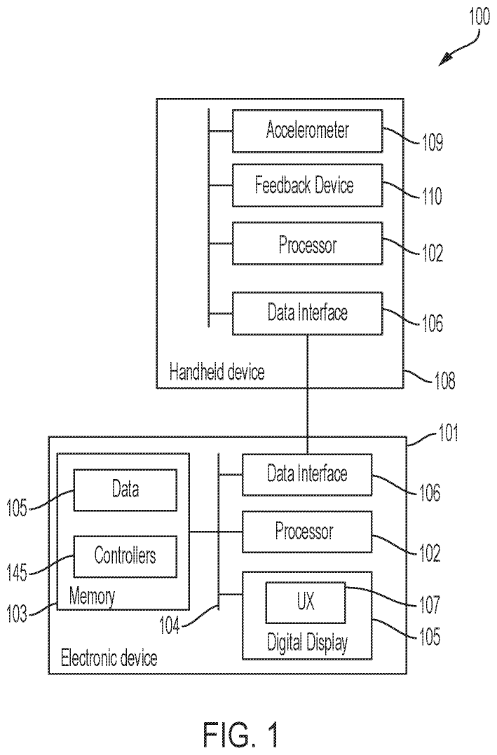

FIG. 1 shows a locomotive style and pace feedback system 100 in accordance with an embodiment.

The system 100 comprises an electronic device 101. The electronic device 101 comprises a processor 102 for processing digital data. A memory device 103 in operable communication with the processor 102 via a system bus 104 is configured for storing digital data including computer program code instructions.

In use, the processor 102 fetches these computer program code instructions and associated data for interpretation and execution of the computational functionality described herein.

The computer program code instructions may be logically divided into a plurality of computer program code instruction controllers 145.

The electronic device 101 further comprises a digital display 105 controlled by the processor 102. The electronic device 101 further comprises a data interface 106 controlled by the processor 102.

A software application may be downloaded via the data interface 106 for installation within memory 103 of the electronic device 101. The controllers 145 of the electronic device 101 may comprise a user interface controller configured to display a user interface 107 using the digital display 105. A haptic overlay may interface the digital display 105 to receive user input gestures in relation to digital data displayed thereby.

The system 100 further comprises a handheld device 108 similarly comprising a processor 102 for processing digital data and having a data interface 106 controlled by the processor 102 for communicating with the electronic device 101. The data interfaces 108 may communicate via a short-range wireless communication protocol, such as the Bluetooth protocol.

Whereas the handheld device 108 may comprise a memory device 103, in embodiments, the handheld device 108 employs low power firmware-based microcontroller architecture.

The handheld device 108 comprises an accelerometer 109 in operable communication with the processor 102 wherein the processor 102 is configured for receiving acceleration signals measured by the accelerometer 109. The accelerometer 109 may comprise the STMicroelectronics LSM6DSV iNEMO™ Inertial Module which is system-in-package featuring a 3-axis digital gyroscope and a 3-axis digital accelerometer, the Murata SCC433T-K03™ Inertial Measurement Units (IMUs) which is a combined high-performance angular rate & accelerometer sensor component based on 3D-MEMS technology, the Analog Devices Inc. ADXL314™ ±200 g 3-Axis Digital Accelerometer which outputs digital data as 16-bit, twos complement, accessible via an SPI, 3- or 4-wire, or an I2C or the like.

The handheld device 108 may further comprise a feedback device 110 configured to provide user feedback.

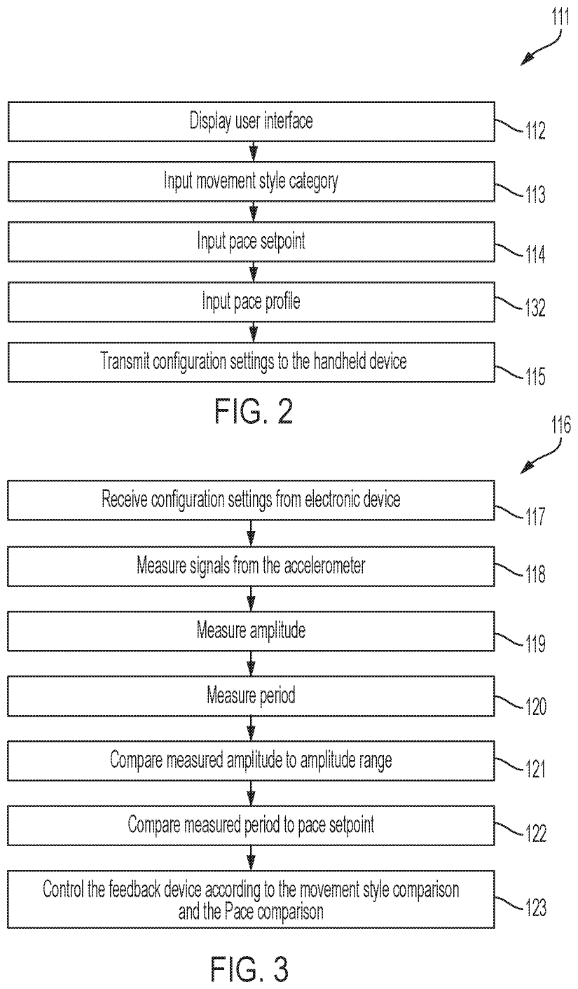

The system 100 is configurable in a configuration mode shown in FIG. 2 wherein the electronic device 101 is used to configure the handheld device 102 with locomotion configuration settings.

The system 100 is further configurable in a feedback mode during exercise shown in FIG. 3 wherein the handheld device provides locomotion feedback according to the locomotion configuration settings.

FIG. 2 shows exemplary configuration mode processing 111 implemented by the processor 102 of the electronic device 101 in accordance with an embodiment.

At step 112, the processor 102 of the electronic device 101 displays a configuration user interface 107.

FIG. 5 shows an exemplary configuration user interface 107 in accordance with an embodiment.

The configuration user interface 107 may comprise a body height input 129. The system 100 may use input body height to determine arm and leg length of a user.

The configuration user interface 107 may further comprise a locomotive style category input 130 to take a locomotive style category at step 113.

In the example provided, the locomotive style category input 130 may have categories such as sprint, power, long distance locomotive styles and the like. As will be described in further detail below, the locomotive style category input 130 is used to determine an arm movement range. In other words, a sprinter would typically move the arms a greater range than that of a long-distance runner.

The configuration user interface 107 may further comprise a pace setpoint input 131 to take a pace setpoint at step 114. The pace setpoint input 131 take the form of a slider and, in the example shown, is used to configure a running pace setpoint of 18 km/h.

In embodiments, the configuration user interface 107 may comprise a pace profile input 133 to take an input pace profile at step 132. A pace profile may be selected from a number of pace profile templates having different shapes. In embodiments, the user interface may be controlled to adjust the shape of the pace profile.

At step 115, the electronic device 101 is configured to transmit configuration settings according to the locomotive style category and the pace setpoint (and, in embodiments, the pace profile) to the handheld device 108 via the respective data interfaces 106 thereof.

FIG. 3 shows exemplary feedback mode processing 116 implemented by the controllers of the handheld device 108.

At step 117, the handheld device 108 receives the configuration settings from the electronic device 117 via the respective data interfaces 106 thereof.

At step 118, and with reference to FIG. 7, the processor 102 of the electronic device 101 measures accelerometer signals 125 from the accelerometer 118. In embodiment shown, a single accelerometer signal 125 is shown. However, the accelerometer 109 may measure acceleration in three axes.

At step 119, the processor 102 of the electronic device 101 uses the signals to determine a measured amplitude 126. The amplitude 126 is representative of the range of motion of a user's arm. In other words, the further the range of motion, the greater the amplitude of acceleration exhibited by the measured amplitude 126.

At step 120, the processor 102 of the electronic device 101 uses the signals to determine a measured period 127. One period is representative of one back-and-forth movement of a user's arm.

At step 121, the processor 102 of the electronic device 101 is configured to compare the measured amplitude 126 to an amplitude range 128 specific to the locomotive style category. With reference to FIG. 7, there is shown the sprint, power and long-distance locomotive style categories each having respective amplitude ranges 128A-C of respective decreasing amplitude. As is shown in FIG. 7, the amplitude range 128C for the long-distance locomotive style expects a lower acceleration amplitude 126 indicative of smaller arm movements as compared to for the range 128A for the sprinter locomotive style.

In other words, a sprint walker would move the arms through a greater movement range as compared to a power walker, and a power walker would similarly move the arms through a greater movement range as compared to a long-distance walker.

The processor 102 of the handheld device 108 may categorise the locomotive style comparison into categories wherein the amplitude 126 is above the amplitude range 128 (i.e., out of target range), wherein the amplitude 126 is within the amplitude range 128 (i.e., within target range) and wherein the amplitude 126 is below the amplitude range 128 (i.e., below target range).

In embodiments, the handheld device 108 is configured to convert the measured amplitude 126 to an arm movement range in accordance with a body dimension configuration setting received from the electronic device 101. For example, the user may input a body height wherein the system 100 infers a typical arm length therefrom using a lookup table or ratio. Using the arm length, the handheld device 108 may convert the measured amplitude 126 to an arm movement range. For example, the handheld device 108 may determine that an acceleration amplitude of 2.7 G the quest to an arm movement range of 30 cm. In embodiments, the user may input an arm length directly using the user interface 107.

At step 122, the processor 102 of the handheld device 108 is configured to compare the measured period to the pace setpoint 122.

The system 100 may be configured to convert the measured period 127 to a measured pace according to a body dimension configuration setting received from the electronic device 101. As alluded to above, the user may input a body height wherein the system 100 determines a typical leg length therefrom using a lookup table or ratio. Using the leg length, the handheld device 108 is able to convert the measured period to measured pace. Alternatively, the user may input a leg length directly.

At step 123, the processor 102 of the handheld device 108 is configured to control the feedback device 110 according to the locomotive style comparison and the pace setpoint comparison. As such, a user is able to determine whether the user is moving correctly (i.e. swinging the arms correctly) according to the chosen locomotive style category and whether the user is walking or running too fast or too slow.

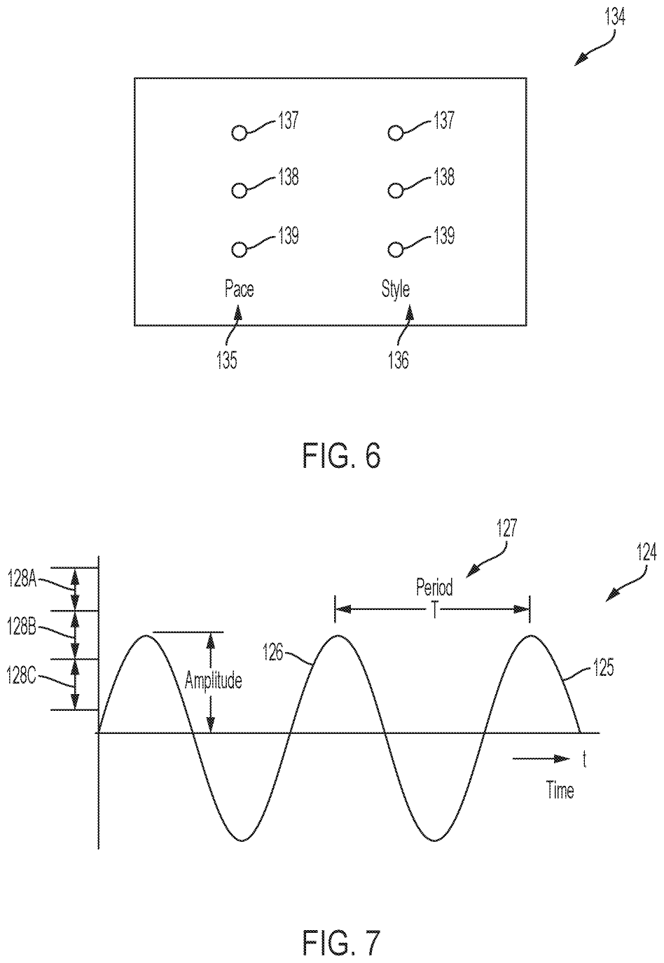

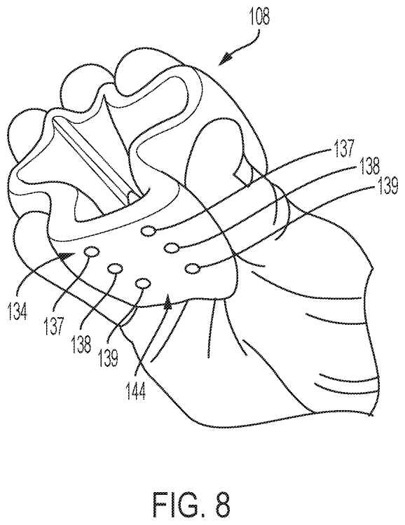

In embodiments, the feedback device 110 comprises a visual indicator 134. FIG. 6 shows an exemplary visual indicator 134 which is designed to be simple to read and non-distracting in use.

In accordance with this example, the visual indicator 134 comprises a series of LEDs to provide feedback. Specifically, the visual indicator 134 may comprise a pace feedback indicator 135 and a separate style feedback indicator 136.

Each indicator 135, 136 may comprise a red above range LED 137, a green in-range LED 138 and an amber below range LED 139.

For example, if the user is running or walking too fast with reference to the pace setpoint, the red above range LED 137 of the pace feedback indicator 137 would be illuminated by the processor 102 of the handheld device 108. Conversely, if the user is walking or running too slow with reference to the pace setpoint, the amber below range LED 139 would illuminate. However, when the user is running within a threshold range of the pace setpoint, the in-range green LED 138 would illuminate.

Similarly, if the user is swinging the arms too much with reference to the locomotive style category, the red above range LED 137 of the style feedback indicator 136 would illuminate.

For example, where the user has selected a long-distance locomotive style, the system 100 may be configured that the arms should move through a range of between 10 and 30 cm. As such, the red above range LED 137 of the style feedback indicator 136 would illuminate if the handheld device 108 measures that the user's arms are moving through a range of 40 cm, the green in-range LED 138 of the style feedback indicator 136 would illuminate if the handheld device 108 measures that the user's arms are moving through a range of 20 cm and the amber below range LED 139 of the style feedback indicator 136 would illuminate if the handheld device 108 measures that the user's arms are moving through a range of 5 cm.

The processor 102 of the handheld device 108 may dynamically adjust the pace setpoint according to the aforedescribed pace profile. The processor 102 may determine the relevant position (X axis) of the pace profile according to time or distance.

For example, the processor 102 of the handheld device 108 may automatically determine a commencement of exercise according to signals received from the accelerometer 109 and start a timer. A current pace from pace profile would be configured according to time wherein, for example, the pace setpoint would gradually increase from 10 km an hour to 18 km an hour for the first five minutes of exercise, plateau to a pace setpoint of 18 km for 30 minutes and then gradually decrease to a pace setpoint of 5 km/h for five minutes.

Alternatively, the processor 102 may measure distance according to the measured period 127, each period 127 indicative of a step from which a distance may be inferred according to the input or calculator leg length of the user.

As such, the pace profile would be configured according to distance as opposed to time and, for example, the pace setpoint would gradually increase from 10 km an hour to 18 km an hour for the first km and plateau at the pace setpoint of 18 km/h for the next 4 km and then gradually decrease to a pace setpoint of 5 km/h for the remaining kilometre.

In embodiments, the system 100 may measure the distance between a pair of handheld devices 108 to infer compliance with the configured locomotive style category. For example, a sprinter would generally hold the arms further apart as compared to a long-distance runner. More specifically, the hands of a long-distance runner may be held closely together in front of the chest whereas the sprinter would hold the hands wider apart either side of the torso.

In a first embodiment, the system 100 may be configured to determine the distance between the handheld devices 108 according to a correlation (phase) of acceleration signals 127 received from each handheld device 108.

For example, the accelerometer 109 may be a triaxial accelerometer configured to three axes so that, for example, a pair of handheld devices held together in front of the chest would generally be moving side-by-side together (i.e., along a frontal axis) as compared to a pair of handheld devices 108 held either side of the torso which would generally move independently along the sagittal axis. As such, phase correlation along the frontal axis may exhibit greater correlation when the handheld devices 109 are held together.

In embodiments, the handheld device 108 may comprise a gyroscopic sensor (not shown) to determine the orientation of the handheld device which may be used to supplement the accelerometer signals or increase the accuracy thereof. Alternatively, as alluded to above, the accelerometer 109 may be a triaxial accelerometer having one axis used to measure acceleration along the frontal axis to determine sideways correlation in movement of the pair of handheld devices 108 and another axis to measure acceleration signals along the sagittal axis to independently measure arm range motion for each arm. In embodiments, the gyroscopic sensor may be referenced to infer which of the three axes measured by the triaxial accelerometer correspond to the frontal sagittal axes respectively.

In alternative embodiments, the system 100 uses a received signal strength indication to measure distance between the handheld devices 108. For example, one handheld device 108 may comprise a wireless transmitter and the other handheld device 108 comprises a wireless receiver. The signal strength indication measured by the wireless receiver may be used to measure a distance between the handheld devices 108.

As such, the visual indicator 134 may further comprise an arm position feedback indicator (not shown) indicative of whether the user is holding the arms too close together or too far apart depending on the configured locomotive style category.

In embodiments, the feedback device 110 of the handheld device 108 comprises a haptic output device which is used as a metronome to provide pulsed haptic feedback to the user.

For example, the haptic output device may comprise an eccentric flywheel, piezoelectric haptic output device or the like which generates haptic feedback (tactile vibration) which is felt by the user when holding the handheld device 108.

The processor 102 of the handheld device 108 may be configured to pulse the haptic output device.

The processor 102 may be configured to pulse the haptic output device at a rate corresponding to the pace setpoint comparison category. For example, if the user is running too fast (i.e., above a pace setpoint range), the haptic output device may emit a short interval of short pulses, if the runner is running within a pace setpoint range, the haptic output device may emit a long interval of short pulses and if the runner is running below the pace setpoint range, the haptic output device may emit a short interval of long pulses.

In alternative embodiments, the processor 102 of the handheld device 108 is configured to pulse the haptic output device at a rate corresponding to the configured pace setpoint. In other words, the pulse may correspond to the footfall rhythm corresponding to the pace setpoint and adjusted according to the leg length of the user. The handheld device 108 may synchronise the pulses with peaks of the accelerometer signals 125 so that the pulses generally correlate with footfall.

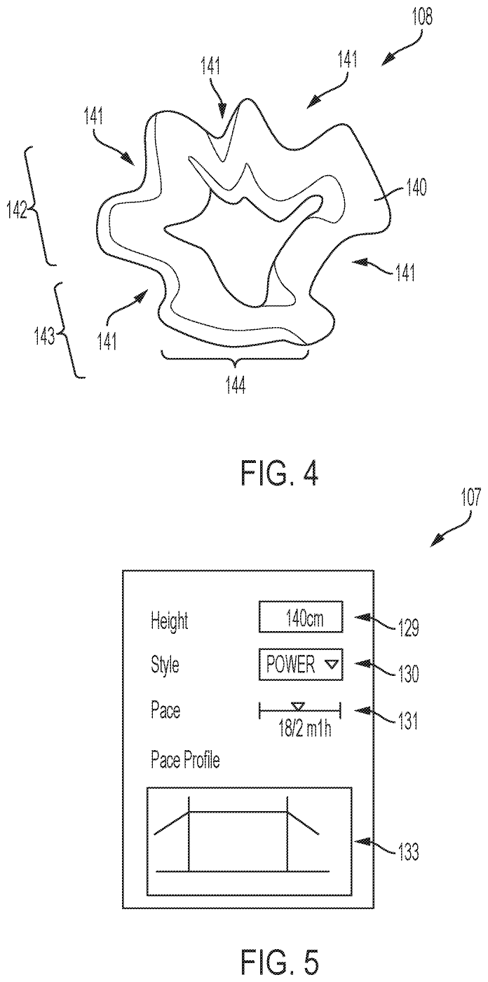

FIG. 4 shows a handheld device 108 which is ergonomically fitting in accordance with an embodiment. In accordance with this embodiment, the handheld device 108 comprises an ergonomically fitting body 140. The body 140 may have an opening therethrough. The opening may allow the body 140 to be squeezed and may reduce the overall weight thereof.

The body 140 defines a plurality of finger indentations 141.

The body 140 may have a wide portion 142 having a plurality of finger indentations 141 for the three middle fingers generally thereacross. The body 140 may further have a narrow portion 143 having opposing finger indentations 141 for opposing grip of the outer fingers (i.e. opposition of the thumb and forefinger).

The body 140 is generally small and lightweight to comfortably fit within the grasp of the fingers.

The body 140 may define a central portion 144 between the opposing finger indentations of the narrow portion 143.

The visual indicator 134 may be located across the central portion 144 for convenient visual positioning.

The foregoing description, for purposes of explanation, used specific nomenclature to provide a thorough understanding of the invention. However, it will be apparent to one skilled in the art that specific details are not required in order to practise the invention. Thus, the foregoing descriptions of specific embodiments of the invention are presented for purposes of illustration and description. They are not intended to be exhaustive or to limit the invention to the precise forms disclosed as obviously many modifications and variations are possible in view of the above teachings. The embodiments were chosen and described in order to best explain the principles of the invention and its practical applications, thereby enabling others skilled in the art to best utilize the invention and various embodiments with various modifications as are suited to the particular use contemplated. It is intended that the following claims and their equivalents define the scope of the invention.

1-31. (canceled)

32. A system comprising a handheld device comprising an ergonomic body comprising a periphery of finger indentations, wherein the body comprises an opening therethrough and is shaped to define a wide portion at one side of the body defining only three middle finger indentations and which narrows to a narrow portion at an opposite side of the body, and wherein opposing sides of the narrow portion define opposing finger indentations for opposing grip of the outer fingers and wherein the opposing finger indentations are symmetric with respect to the three middle finger indentations.

33. The system as claimed in claim 32, further comprising:
an electronic device comprising a processor, a digital display controlled by the processor, a data interface controlled by the processor and a memory device in operable communication with the processor, the memory device comprising computer program code instruction controllers executed by the processor;
the handheld device comprising a processor, a data interface controlled by the processor, an accelerometer in operable communication with the processor, a feedback device controlled by the processor, the processor executing computer program code instruction controllers, wherein the system is configured to operate in:
a configuration mode wherein the controllers of the electronic device:
control a user interface displayed by the digital display to set:
a locomotive style category; and
a pace setpoint,
control the data interface of the electronic device to transmit configuration settings according to the locomotive style category and the pace setpoint to the handheld device via the data interface of the handheld device; and
a feedback mode wherein the controllers of the handheld device:
measure signals from the accelerometer to determine:
measured amplitude; and
measured period; and
compare the measured amplitude to an amplitude range of the locomotive style category to calculate a locomotive style comparison;
compare the measured period to the pace setpoint to calculate a pace setpoint comparison; and
control the feedback device according to the locomotive style comparison and the pace setpoint comparison.

34. The system as claimed in claim 33, wherein the locomotive style comparison categorises whether the amplitude is above the amplitude range, wherein the amplitude is within the amplitude range and wherein the amplitude is below the amplitude range.

35. The system as claimed in claim 33, wherein the pace setpoint comparison categorises whether the measured period is above a period range, wherein the measured period is within the period range and wherein the measured period is below the period range.

36. The system as claimed in claim 33, wherein the controllers of the handheld device convert the measured amplitude to an arm movement range in accordance with a configuration setting received from the electronic device.

37. The system as claimed in claim 36, wherein the configuration setting comprises body height and wherein the system is configured to convert the body height to an arm length.

38. The system as claimed in claim 36, wherein the configuration setting comprises arm length.

39. The system as claimed in claim 33, wherein the controllers of the handheld device convert the measured period to a measured pace in accordance with a configuration setting received from the electronic device.

40. The system as claimed in claim 39, wherein the configuration setting comprises body height and wherein the system is configured to convert the body height to a leg length.

41. The system as claimed in claim 39, wherein the configuration setting comprises leg length.

42. The system as claimed in claim 33, wherein, in the configuration mode, the controllers of the electronic device:
control the user interface displayed by the digital display to configure a pace profile; and
control the data interface of the electronic device to transmit the configuration settings further according to the pace profile to the handheld device via the data interface of the handheld device; and
in the feedback mode, the controllers of the handheld device:
calculate a variable pace setpoint according to the pace profile; and
compare the measured period to the variable pace setpoint to calculate the pace setpoint comparison.

43. The system as claimed in claim 42, wherein the controllers of the handheld device are configured to calculate the variable pace according to time.

44. The system as claimed in claim 43, wherein the controllers of the handheld device are configured to analyse the signals from the accelerometer to detect motion to commence a timer to measure the time.

45. The system as claimed in claim 42, wherein the controllers of the handheld device are configured to calculate the variable pace according to distance.

46. The system as claimed in claim 43, wherein the controllers of the handheld device are configured to calculate the distance according to the signals from the accelerometer.

47. The system as claimed in claim 33, wherein the feedback device comprises a visual indicator controlled by the controllers of the electronic device to:
visually display a locomotive style comparison category; and
visually display a pace setpoint comparison category.

48. The system as claimed in claim 47, wherein the visual indicator comprises:
an above range indicator;
an in-range indicator; and
a below range indicator for each of the locomotive style comparison category and the pace setpoint comparison category.

49. The system as claimed in claim 33, wherein the system comprises a pair of handheld devices and wherein, in the feedback mode, the system is configured to determine a distance between the handheld devices to further calculate an arm position locomotive style comparison.

50. The system as claimed in claim 49, wherein the system is configured to determine a distance between the handheld devices according to a received signal strength indication of a receiver of one of the handheld devices receiving a signal from a transmitter of another of the handheld devices.

51. The system as claimed in claim 49, wherein the system is configured to determine the distance between the handheld devices according to acceleration signal phase of the handheld devices.

52. The system as claimed in claim 51, wherein each handheld device comprises a triaxial accelerometer and wherein the acceleration signal phase is measured from frontal axes acceleration signals thereof.

53. The system as claimed in claim 47, wherein the system comprises a pair of handheld devices and wherein, in the feedback mode, the system is configured to determine a distance between the handheld devices to further calculate an arm position locomotive style comparison and wherein the visual indicator is further configured to visually display an arm position locomotive style comparison category.

54. The system as claimed in claim 33, wherein the controller of the handheld device is configured to autodetect exercise according to the signals from the accelerometer.

55. The system as claimed in claim 33, wherein the feedback device comprises a haptic output device and wherein the controllers of the handheld device are configured to pulse the haptic output device.

56. The system as claimed in claim 55, wherein the controllers of the handheld device are configured to pulse the haptic output device at a rate corresponding to the pace setpoint comparison.

57. The system as claimed in claim 55, wherein the controllers of the handheld device are configured to pulse the haptic output device at a rate corresponding to the pace setpoint.

58. The system as claimed in claim 57, wherein the controllers of the handheld device are configured to correlate pulses with peaks of the signals received from the accelerometer of the handheld device.

59. The system as claimed in claim 47, wherein the narrow portion defines a central portion between the opposing finger indentations and wherein the visual indicator is located at the central portion.
